(19) United States
(12) Patent Application Publication
(10) Pub. No.: US 2019/0177689 A1
(43) Pub. Date: Jun. 13, 2019

(54) ISOLATION OF OOGONIAL STEM CELLS

(71) Applicants: Hossein Baharvand, Tehran (IR); Maryam Saber, Tehran (IR); Fereshteh Esfandiari Nezhad, Tehran (IR)

(72) Inventors: Hossein Baharvand, Tehran (IR); Maryam Saber, Tehran (IR); Fereshteh Esfandiari Nezhad, Tehran (IR)

(73) Assignee: Royan Institute, Tehran (IR)

(21) Appl. No.: 16/214,340

(22) Filed: Dec. 10, 2018

Related U.S. Application Data
(60) Provisional application No. 62/597,446, filed on Dec. 12, 2017.

(51) Int. Cl. C12N 5/075

(52) U.S. Cl. CPC C12N 5/0609; C12N 2501/11; C12N 2501/115; C12N 2533/00; C12N 2501/235; C12N 2513/00; C12N 2501/13

(57) ABSTRACT

A method for isolating oogonial stem cells (OSCs) including forming an ovarian cell suspension from an ovary, forming ovaroids by culturing the ovarian cell suspension in a three-dimensional culture, and migrating the OSCs around the ovaroids by culturing the ovaroids on a mouse embryonic fibroblast (MEF)-coated plate.

### CROSS-REFERENCE TO RELATED APPLICATION

This application claims the benefit of priority from pending U.S. Provisional Patent Application Ser. No. 62/597,446, filed on Dec. 12, 2017, and entitled “OOGONIAL CELL ISOLATION VIA ORGANOID APPROACH,” which is incorporated herein by reference in its entirety.

### SPONSORSHIP STATEMENT

This application has been sponsored by Iran Patent Center, which does not have any rights in this application.

### TECHNICAL FIELD

The present disclosure generally relates to oogonial stem cells (OSCs), particularly to a method for isolating OSCs, and more particularly to a method for isolating OSCs via forming ovarian organoids (ovaroids).

### BACKGROUND

Discovery of oogonial stem cells (OSCs) in adult ovaries and their contribution to forming new offspring has opened new ways to study female reproductive biology and develop advanced therapies for ovarian diseases. Despite the crucial importance of OSCs in reproductive medicine, a feasible method for isolating these cells has remained elusive due to the relative rarity of OSCs.

Conventional methods for isolating OSCs entail utilizing genii cell-specific markers in fluorescence-activated cell sorting (FACS) and magnetic-activated cell sorting (MACS) methods. However, these methods are time-consuming and have various shortcomings such as low efficiency, low reproducibility, low expansion ability, and high technical difficulty. Accordingly, there is a need for a faster and more cost-effective method with technical simplicity, high reproducibility, and feasibility for isolating OSCs from both mouse and human adult ovaries.

### SUMMARY

This summary is intended to provide an overview of the subject matter of the exemplary embodiments of the present disclosure, and is not intended to identify essential elements or key elements of the subject matter, nor is it intended to be used to determine the scope of the claimed implementations. The proper scope of the exemplary embodiments of the present disclosure may be ascertained from the claims set forth below in view of the detailed description below and the drawings.

In one general aspect, the present disclosure describes an exemplary method for isolating oogonial stem cells (OSCs). The exemplary method may include forming an ovarian cell suspension from an ovary, forming an ovarian organoid (ovaroid) by culturing the ovarian cell suspension in a three-dimensional culture and migrating the OSCs around the ovaroid by culturing the ovaroids on a mouse embryonic fibroblast (MEF)-coated plate.

The above general aspect may include one or more of the following features. In some exemplary embodiments, culturing the ovarian cell suspension in the three-dimensional culture may include culturing the ovarian cell suspension using one of an agarose-coated plate, a hanging drop, or a microwell. In some exemplary implementations, forming the ovaroid by culturing the ovarian cell suspension may include culturing the ovarian cell suspension for a period of time between about 2 days and about 3 days. In some exemplary embodiments, forming the ovaroid by culturing the ovarian cell suspension may include culturing the ovarian cell suspension with a density between about 2500 cell/cm² and about 500000 cell/cm².

According to some exemplary implementations, forming the ovarian cell suspension from the ovary may include removing mature oocytes from the ovarian cell suspension using a filter with a filter mesh about 40 μm. In some exemplary implementations, forming the ovaroid by culturing the ovarian cell suspension may include culturing the ovarian cell suspension in a medium including a growth factor.

According to some exemplary embodiments, the growth factor may include one of epidermal growth factor (EGF), basic fibroblast growth factor (bFGF), glial cell line-derived neurotrophic factor (GDNF), leukemia inhibitory factor (LIF), or combinations thereof. According to some exemplary embodiments, the ovaroid may have a diameter between about 50 μm and about 100 μm. In some exemplary embodiments, the ovaroid may have a round shape or an oval shape.

According to some exemplary implementations, migrating the OSCs around the ovaroid by culturing the ovaroid on the MEF-coated plate may include culturing the ovaroid with a density of about 1 ovaroid/cm² on the MEF-coated plate. In some exemplary embodiments, migrating the OSCs around the ovaroid by culturing the ovaroid on the MEF-coated plate may include culturing the ovaroid for a time period between about 1 week and about 3 weeks.

According to some exemplary implementations, migrating the OSCs around the ovaroid by culturing the ovaroid on the MEF coated plate may include culturing the ovaroid in a medium including a growth factor. In some exemplary embodiments, the growth factor may include one of epidermal growth factor (EGF), basic fibroblast growth factor (bFGF), glial cell line-derived neurotrophic factor (GDNF), leukemia inhibitory factor (LIF), or combinations thereof. According to some exemplary embodiments, the OSCs may have a genii cell-specific marker including Vasa, Dazl, Oct4, Fragilis, Stella, and Blimp-1. In some exemplary embodiments, the OSCs may include one of mouse OSCs (mOSCs) or human OSCs (hOSCs).

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates a method for isolating oogonial stem cells (OSCs), consistent with one or more exemplary of the present disclosure.

FIG. 2A illustrates a phase-contrast image of a mouse ovarian cell suspension, consistent with one or more exemplary embodiments of the present disclosure.

FIG. 2B illustrates a phase-contrast image of ovaroids after 2 culture days of an ovarian cell suspension, consistent with one or more exemplary embodiments of the present disclosure.

FIG. 2C illustrates a histological image of an ovaroid by hematoxylin and eosin (H&E) staining, consistent with one or more exemplary embodiments of the present disclosure.

FIG. 2D illustrates a histological image of a magnified portion of an ovaroid by hematoxylin and eosin (H&E) staining, consistent with one or more exemplary embodiments of the present disclosure.

FIG. 2E illustrates a phase-contrast image of mouse oogonial stem cells (mOSCs) derived from re-plated ovaroid after 3 days, consistent with one or more exemplary embodiments of the present disclosure.

FIG. 2F illustrates a phase-contrast image of mouse oogonial stem cells (mOSCs) derived from re-plated ovaroid after 12 days, consistent with one or more exemplary embodiments of the present disclosure.

FIG. 2G illustrates a phase-contrast image of mouse oogonial stem cells (mOSCs) derived from re-plated ovaroid after 21 days, consistent with one or more exemplary embodiments of the present disclosure.

FIG. 2H illustrates a phase-contrast image of a magnified portion of mouse oogonial stem cells (mOSCs) derived from re-plated ovaroid after 21 days, consistent with one or more exemplary embodiments of the present disclosure.

FIG. 3A illustrates phase-contrast images of expanded mouse oogonial stem cells (mOSCs) after 5, 10, 25, and 60 passages on mouse embryonic fibroblast (MEF)-coated plates, consistent with one or more exemplary embodiments of the present disclosure.

FIG. 3B illustrates proliferation pattern of mouse oogonial stem cells (mOSCs) during 210 days, consistent with one or more exemplary embodiments of the present disclosure.

FIG. 3C illustrates a phase-contrast image of mouse oogonial stem cells (mOSCs) after thawing, consistent with one or more exemplary embodiments of the present disclosure.

FIG. 3D illustrates fluorescence-activated cell sorting (FACS) results for the viability of mouse oogonial stem cells (mOSCs) before and after cryopreservation after passage 20, consistent with one or more exemplary embodiments of the present disclosure.

FIG. 4A illustrates immunofluorescent images of mouse ovaroid sections, consistent with one or more exemplary embodiments of the present disclosure.

FIG. 4B illustrates results of reverse transcription polymerase chain reaction (RT-PCR) analysis of ovary versus ovaroids on day 2 of culture, consistent with one or more exemplary embodiments of the present disclosure.

FIG. 5A illustrates immunofluorescent images of mouse oogonial stem cells (mOSCs) for detection of oocyte-specific markers, consistent with one or more exemplary embodiments of the present disclosure.

FIG. 5B illustrates results of reverse transcription polymerase chain reaction (RT-PCR) analysis of mouse oogonial stem cells (mOSCs) for detection of germ cell-specific markers after 2 weeks of the ovaroid culture, consistent with one or more exemplary embodiments of the present disclosure.

FIG. 5C illustrates immunofluorescent images of the mouse oogonial stem cells (mOSCs) for detection of germ cell-specific markers and stem cell-specific marker after 12 passages as a long-term culture, consistent with one or more exemplary embodiments of the present disclosure.

FIG. 5D illustrates percentages of Vasa- and Ki67-positive mouse oogonial stem cells (mOSCs) on day 21 of culture before any passage and after 12 passages as a long-term culture, consistent with one or more exemplary embodiments of the present disclosure.

FIG. 6A illustrates a phase-contrast image of a human ovarian cell suspension, consistent with one or more exemplary embodiments of the present disclosure.

FIG. 6B illustrates a phase-contrast image of human ovaroids after 2 culture days of human ovarian cell suspension, consistent with one or more exemplary embodiments of the present disclosure.

FIG. 6C illustrates a histological image of an ovaroid by hematoxylin and eosin (H&E) staining, consistent with one or more exemplary embodiments of the present disclosure.

FIG. 7A illustrates phase-contrast images of expanded human oogonial stem cells (hOSCs) after 30 days culture, 1 passage, 3 passages, 5 passages, 6 passages, and 7 passages on MEF-coated plates, consistent with one or more exemplary embodiments of the present disclosure.

FIG. 7B illustrates growth curve of human oogonial stem cells (hOSCs), consistent with one or more exemplary embodiments of the present disclosure.

FIG. 8 illustrates immunofluorescent images of human ovaroid sections for detection of genii cell-specific markers, consistent with one or more exemplary embodiments of the present disclosure.

FIG. 9A illustrates immunofluorescent images of human oogonial stem cells (hOSCs) for detection of germ cell-specific markers, consistent with one or more exemplary embodiments of the present disclosure.

FIG. 9B illustrates immunofluorescent images of human oogonial stem cells (hOSCs) for Vasa and Ki67 markers, consistent with one or more exemplary embodiments of the present disclosure.

FIG. 9C illustrates the percentage of Vasa-positive and Ki67-positive human oogonial stern cells (hOSCs) after 6 months as a long-term culture, consistent with one or more exemplary embodiments of the present disclosure.

FIG. 10A illustrates phase-contrast images of mouse oocyte-like cells (mOLCs), consistent with one or more exemplary embodiments of the present disclosure.

FIG. 10B illustrates ratio of oocyte-like cells and oogonial stern cells (OLCs/OSCs) in spontaneous differentiation and directed differentiation, consistent with one or more exemplary embodiments of the present disclosure.

FIG. 10C illustrates size distribution and a number of formed oocyte-like cells (OLCs) in spontaneous differentiation and directed differentiation, consistent with one or more exemplary embodiments of the present disclosure.

FIG. 10D illustrates mRNA expression levels of oocyte-specific markers in spontaneously differentiated oocyte-like cells (OLCs), consistent with one or more exemplary embodiments of the present disclosure.

FIG. 10E illustrates immunofluorescence images of spontaneously differentiated oocyte-like cells (OLCs) for detection of germ cell-specific markers and oocyte-specific markers, consistent with one or more exemplary embodiments of the present disclosure.

FIG. 11A illustrates a phase-contrast image of human oocyte-like cells (hOLCs), consistent with one or more exemplary embodiments of the present disclosure.

FIG. 11B illustrates immunofluorescence images of spontaneously differentiated oocyte-like cells (OLCs) for detection of germ cell-specific markers and oocyte-specific markers, consistent with one or more exemplary embodiments of the present disclosure.

FIG. 12A illustrates histological images of a normal ovary, busulfan treated ovary, and an ovary with transplanted mOSCs by hematoxylin and eosin (H&E) staining, consistent with one or more exemplary embodiments of the present disclosure.

FIG. 12B illustrates histological images of magnified portions of different ovaries after mouse oogonial stem cells (mOSCs) transplantation by hematoxylin and eosin (H&E) staining, consistent with one or more exemplary embodiments of the present disclosure.

FIG. 12C illustrates histological images of follicles of mouse oogonial stem cells (mOSCs) transplanted ovary at various stages of maturation after immunohistochemistry (IHC) staining, consistent with one or more exemplary embodiments of the present disclosure.

FIG. 12D illustrates gel electrophoresis profile for polymerase chain reaction (PCR) products of offspring derived from transplanted green fluorescence protein (GFP)-positive mouse oogonial stem cells (mOSCs), consistent with one or more exemplary embodiments of the present disclosure.

FIG. 12E illustrates gel electrophoresis profile for polymerase chain reaction (PCR) products of F2 progeny derived from transplanted green fluorescence protein (GFP)-positive mouse oogonial stem cells (mOSCs), consistent with one or more exemplary embodiments of the present disclosure.

FIG. 12F illustrates immunofluorescence image of human ovarian tissue pieces with injected hOSCs after two weeks of xenotransplantation into nude female mice, consistent with one or more exemplary embodiments of the present disclosure.

FIG. 13 illustrates microscopic images for embryo development of an oogonial stem cells (OSCs)-derived oocyte and a host-derived oocyte, consistent with one or more exemplary embodiments of the present disclosure.

FIG. 14 illustrates histological images of the mOSCs-injected sites of the neck area of mice stained with hematoxylin and eosin (H&E) staining, consistent with one or more exemplary embodiments of the present disclosure.

FIG. 15 illustrates levels of β-estradiol (E2) and inhibin produced by ovaroids in presence or absence of follicle-stimulating hormone (FSH) and luteinizing hormone (LH), consistent with one or more exemplary embodiments of the present disclosure.

FIG. 16 illustrates alkaline phosphatase staining (AP) of ovaroid-derived oogonial stem cells (OSCs) and embryonic stem cells (ESCs), consistent with one or more exemplary embodiments of the present disclosure.

### DETAILED DESCRIPTION

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

The use of oogonial stem cells (OSCs) has been postulated to aid in the treatment of women unable to produce fertilizable oocytes. However, a feasible method for isolation of OSCs has not been within grasp. Recent approaches in three-dimensional (3D) cultures have led to the successful generation of organoids which provides a reliable niche that supports stern cell self-renewal and differentiation. Therefore, producing an ovarian organoid (hereinafter named as “ovaroid”) would provide a suitable environment for proliferation and emergence of OSCs.

Disclosed herein is an exemplary method for isolating OSCs from either mouse or human ovaries in an efficient and reproducible manner by employing ovarian organoids (ovaroids). The ovaroid provides us with an invaluable source of functional OSCs with high potentials for in-vitro expansion, while OSCs that may be acquired from fluorescent-and magnetic-based methods are not expandable or show poor expansion ability in-vitro. This expandable source of OSCs allows us to study germ cell development and ovarian diseases, and for their use in clinic for infertile women with low-quality oocytes.

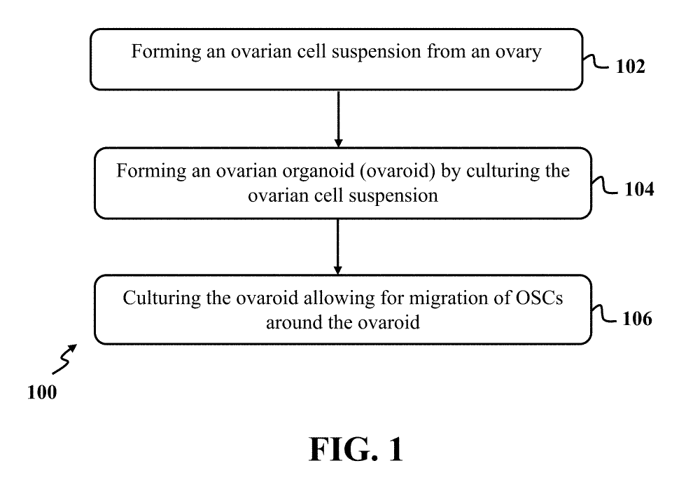

FIG. 1 shows exemplary method 100 for isolating OSCs, consistent with one or more exemplary embodiments of the present disclosure. Method 100 may include forming an ovarian cell suspension from an ovary (step 102), forming an ovaroid by culturing the ovarian cell suspension (step 104) and migrating of OSCs around the ovaroid by culturing the ovaroid (step 106).

Step 102 may include forming the ovarian cell suspension using the ovary. In some exemplary implementations, forming the ovarian cell suspension using the ovary may include washing the ovary, dissecting the washed ovary, extracting ovarian cells by digesting the dissected ovary, and re-suspending the extracted ovarian cells in culture medium. In some exemplary embodiments, washing the ovary may include washing the ovary in Hank's balanced salt solution (HBSS) containing antibiotics.

In some exemplary embodiments, dissecting the ovary may include mincing the ovary using a sterile surgical instrument. In some exemplary embodiments, extracting ovarian cells by digesting the dissected ovary may include enzymatic digestion of the dissected ovary. In one or more exemplary embodiments, enzymatic digestion of the dissected ovary may include enzymatic digestion of the dissected ovary using a collagenase/DNase solution at a temperature of about 37° C. In some exemplary embodiments, washing the extracted ovarian cells may be done prior to re-suspending the extracted ovarian cells in culture medium.

In some exemplary embodiments, the ovarian cell suspension may include oogonial stem cells and ovarian somatic cells. In some exemplary implementations, forming the ovarian cell suspension from the ovary may further include removing mature oocytes from the ovarian cell suspension. In one or more exemplary embodiments, removing the mature oocytes from the ovarian cell suspension may be done using a filter with a filter mesh about 40 μm.

Step 104 may include forming the ovaroid by culturing the ovarian cell suspension. In some exemplary embodiments, the ovaroids may be ovarian cell aggregates with a diameter between about 50 μm and about 100 μm. In some exemplary embodiments, the ovaroids may have a round shape or an oval shape. In one or more exemplary embodiments, the ovarian cell suspension may be cultured in a suspension culture. In some exemplary implementations, forming the ovaroid by culturing the ovarian cell suspension may include culturing the ovarian cell suspension in a three-dimensional culture using one of an agarose-coated plate, a hanging drop, or a microwell.

In some exemplary embodiments, forming the ovaroid by culturing the ovarian cell suspension may include culturing the ovarian cell suspension for a period of time between about 2 days and about 3 days. In some exemplary embodiments, forming the ovaroid by culturing the ovarian cell suspension may include culturing the ovarian cell suspension with a density between about 2500 cell/cm² and about 500000 cell/cm².

In some exemplary embodiments, forming the ovaroid by culturing the ovarian cell suspension may include culturing the ovarian cell suspension in a culture medium including a basic medium supplemented with serum, knock-out serum replacement (KSR), non-essential amino acids, antibiotics, sodium pyruvate, β-mercaptoethanol, L-glutamine, N2 supplement, and a growth factor. In some exemplary embodiments, the basic medium may include one of minimum essential medium Eagle alpha (α-MEM), high glucose Dulbecco's modified Eagle's medium (H-DMEM). Dulbecco's modified Eagle medium/nutrient mixture F-12(DMEM-F12), or serum-free medium. In one or more exemplary embodiments, the serum may include fetal bovine serum (FBS).

In some exemplary embodiments, the growth factor may include one of epidermal growth factor (EGF), basic fibroblast growth factor (bFGF), glial cell line-derived neurotrophic factor (GDNF), leukemia inhibitory factor (LIF), stem cell factor (SCF), or combinations thereof. In one or more exemplary embodiments, EGF may be present in the culture medium with a concentration of about 10 ng/ml. In one or more exemplary embodiments, bFGF may be present in the culture medium with a concentration of about 1 ng/ml. In one or more exemplary embodiments, GDNF may be present in the culture medium with a concentration of about 40 ng/ml. In one or more exemplary embodiments, LIF may be present in the culture medium with a concentration of about 10³ units/ml. In one or more exemplary embodiments, the SCF may be present in the culture medium with a concentration less than about 50 ng/ml.

Step 106 may include migrating the OSCs around the ovaroid by culturing the ovaroid. In some exemplary embodiments, culturing the ovaroid allows the OSCs to radially migrate around the ovaroid. In some exemplary embodiments, the OSCs may be migrated from the ovaroids about three days after culturing the ovaroid.

In some exemplary implementations, migrating the OSCs around the ovaroid by culturing the ovaroid may include culturing the ovaroid in an adherent culture. In some exemplary embodiments, migrating the OSCs around the ovaroid by culturing the ovaroid may include culturing the ovaroid on a mouse embryonic fibroblast (MEF)-coated plate. In some exemplary embodiments, culturing the ovaroid may include culturing the ovaroid with a density of 1 ovaroid/cm².

In some exemplary embodiments, migrating the OSCs around the ovaroid by culturing the ovaroid may include culturing the ovaroid for a time period between about 1 week and about 3 weeks. In some exemplary embodiments, migrating the OSCs around the ovaroid by culturing the ovaroid may include culturing the ovaroid in a culture medium including a basic medium supplemented with serum, knock-out serum replacement (KSR), non-essential amino acids, antibiotics, sodium pyruvate, β-mercaptoethanol, L-glutamine, N2 supplement, and a growth factor. In some exemplary embodiments, the basic medium may include one of minimum essential medium Eagle alpha (α-MEM), high glucose Dulbecco's modified Eagle's medium (H-DMEM). Dulbecco's modified Eagle medium/nutrient mixture F-12 (DMEM-F12), or serum-free medium. In one or more exemplary embodiments, the serum may include fetal bovine serum (FBS).

In some exemplary embodiments, the growth factor may include one of epidermal growth factor (EGF), basic fibroblast growth factor (bFGF), glial cell line-derived neurotrophic factor (GDNF), leukemia inhibitory factor (LIF), stem cell factor (SCF), or combinations thereof. In one or more exemplary embodiments, EGF may be present in the culture medium with a concentration of about 10 ng/ml. In one or more exemplary embodiments, bFGF may be present in the culture medium with a concentration of about 1 ng/ml. In one or more exemplary embodiments, GDNF may be present in the culture medium with a concentration of about 40 ng/ml. In one or more exemplary embodiments, LIF may be present in the culture medium with a concentration of about 10³ units/ml. In one or more exemplary embodiments, the SCF may be present in the culture medium with a concentration less than about 50 ng/ml.

In some exemplary embodiments, during culturing the ovaroids, OSCs as ovaroid-derived may undergo mitosis and form grape-like clusters. In some exemplary embodiments, the OSCs may have a germ cell-specific marker including Vaza, Dazl, OCT4, Fragilis, Stella, and Blimp-1. In some exemplary embodiments, the OSCs may include one of mouse OSCs (mOSCs) or human OSCs (hOSCs). In some exemplary embodiments, the OSCs may have a diameter between about 5 μm and about 10 μm.

### EXAMPLES

### Example 1: Isolation Of Mouse Oogonial Stem Cells

In this example, mOSCs were isolated through steps of forming an ovarian cell suspension using an adult mouse ovary, forming ovaroids by culturing the ovarian cell suspension and migrating mOSCs around the ovaroids by culturing the ovaroids.

Adult 6-8 week-old female NMRI mice were used for ovarian cells isolation from ovarian tissues. Also, green fluorescent protein (GFP)-positive OSCs were isolated from ovarian tissues of transgenic mice which expressed GFP under chicken beta-actin promoter and cytomegalovirus enhancer (pCAG). All animals were maintained in a 12-hour light-dark cycle with access to water and standard chow ad libitum.

At first, the ovarian cell suspension was formed using the adult mouse ovary through washing, dissecting, and digesting the ovary. In each experiment, ovarian tissues of 4 mice were collected and fat pad, bursa, and oviduct were removed from each of the mice ovarian tissues carefully. The ovaries were dissected by mincing and were washed twice in Hank's balanced salt solution (HBSS) containing 1% penicillin and streptomycin.

After that, dissected ovarian tissues underwent an enzymatic digestion involving a gentle agitation with collagenase IV with a concentration of about 800 U/ml and DNase I with a concentration of about 1 μg/ ml at a temperature of about 37° C. in a water bath for a time period of about 10 minutes. After enzymatic digestion, the supernatant of the digested ovarian tissues was collected into a new 15-ml conical tube on ice and about 10 ml of HBSS was added to a conical tube.

Remaining ovarian tissue parts were re-suspended in a fresh, warm enzyme solution of collagenase IV and DNase I, and underwent enzymatic digestion until most of the ovarian tissues were dispersed. Since germ cells are sensitive to enzymes, supernatant was collected after each enzymatic digestion and performed enzymatic digestion on remaining ovarian tissue fragments.

Finally, collected supernatants from all enzymatic digestions were filtered through a 40-μm nylon mesh to remove mature oocytes and centrifuged at speed of about 300 g for a time period of about 5 minutes. Then, the supernatant was removed carefully and the ovarian cell suspension was formed by re-suspending clumps of cells in a culture medium. While mature oocytes have inhibitory effects in the ovary, this inhibition was eliminated by removing the mature oocytes from the ovarian cells. Therefore, a permissive and suitable niche for the survival and proliferation of OSCs was provided.

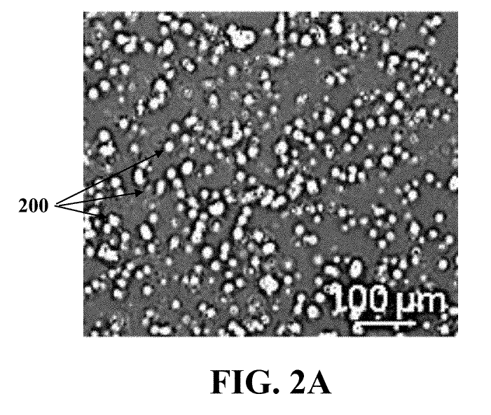

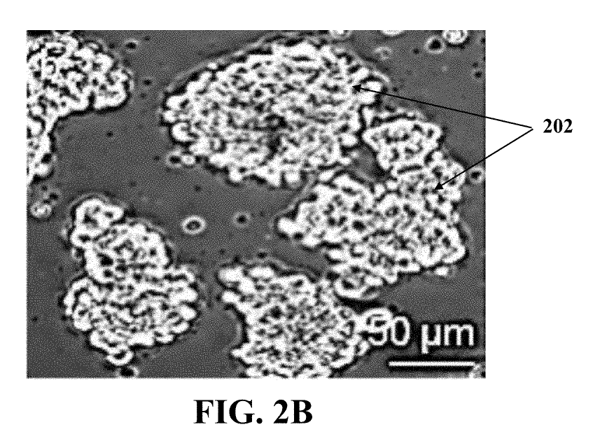

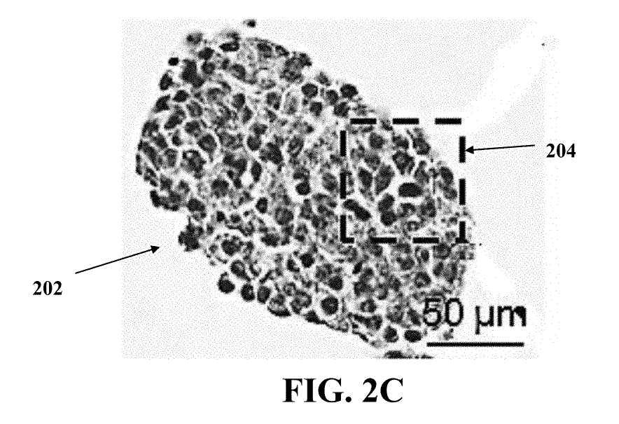

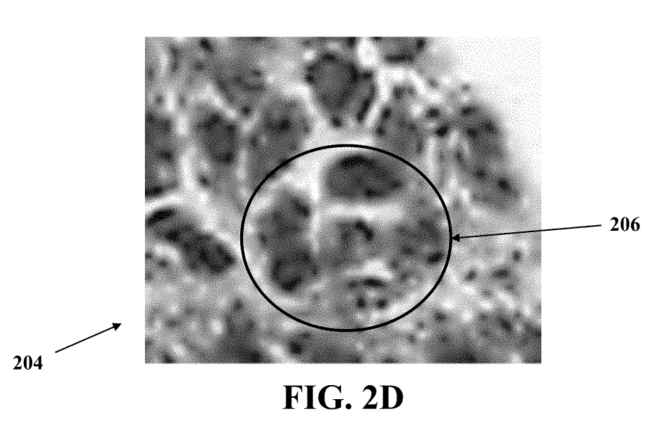

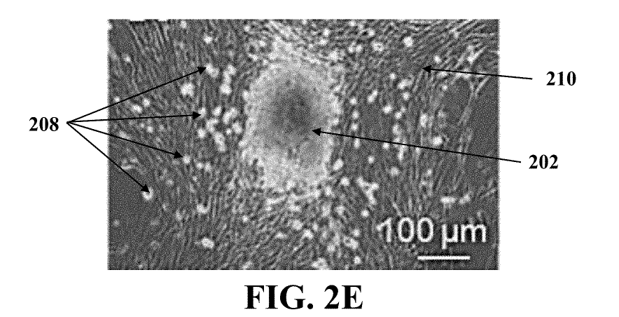

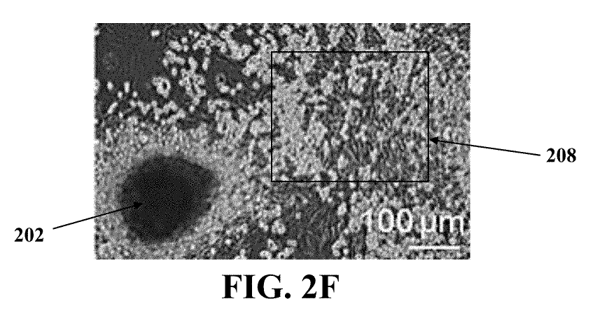

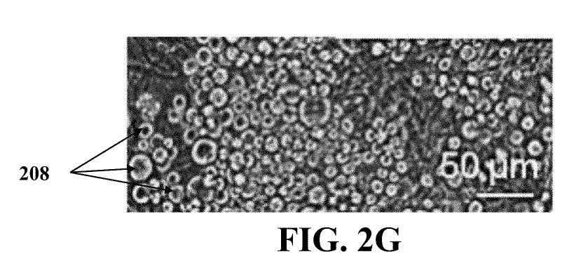

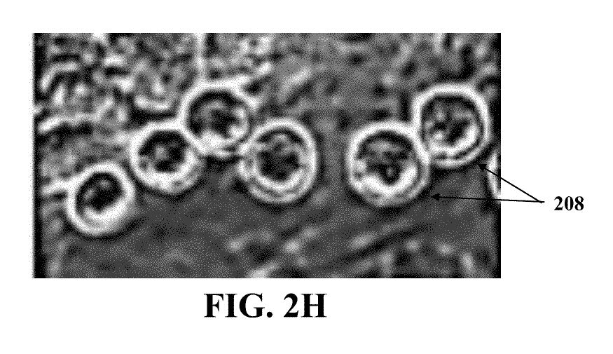

FIG. 2A shows a phase-contrast image of a mouse ovarian cell suspension, consistent with one or more exemplary embodiments of the present disclosure. Referring to FIG. 2A, the mouse ovarian cell suspension may include a homogeneous suspension of mouse ovarian cells 200 without any aggregations.

In the next step, ovaroids were formed by culturing the ovarian cell suspension. The ovarian cell suspension was cultured under floating conditions on agarose-coated plates as a three-dimensional (3D) culture. Cells of the ovarian cell suspension were seeded on plates pre-coated with about 1% agarose and incubated at a temperature of about 37° C. and at 5% CO₂ atmosphere in a culture medium. After 2 or 3 days of cell culture, round or oval aggregates as ovaroids, with a diameter between about 50 μm and about 100 μm, were formed.

The culture medium was α-MEM supplemented with 10% FBS, 1 mM sodium pyruvate, 1 mM nonessential amino acids, 2 mM L-glutamine, 1× penicillin-streptomycin, 0.1 mM β-mercaptoethanol, 1× concentrated N2 supplement, 10³ units/ml leukemia inhibitory factor (LIF), 10 ng/ml recombinant human epidermal growth factor (rhEGF), 1 ng/ml basic fibroblast growth factor (bFGF), and 40 ng/ml glial cell-derived neurotrophic factor (GDNF).

Cell seeding was optimized using different initial cell density on agarose-coated plates between about 1×10⁵ cells per 3 cm² and about 5×10⁵ cells per 3 cm². After optimization, it was found that the density of about 3×10⁵ cells per 3 cm² plate is suitable for ovaroid formation at culture time period of about 2 or 3 days. Seeding cells less than about 3×10⁵ per 3 cm² decreased both size and number of aggregates which underwent gradual degeneration. Furthermore, aggregate formation with higher cellular density lead to the aggregates joining together in 24 hours which led to the formation of dense structures.

The ovaroids included both germ cells and somatic cells, and communication between germ cells and somatic cells are critical for stem cell maintenance and genu cell proliferation. Therefore, the ovaroids served as a well-qualified niche for OSCs because ovaroids as a 3D culture mimic physiological conditions in-vitro by supporting intensive communication between cells and by providing biomechanical and biochemical cues that mediate intercellular functions.

FIG. 2B shows a phase-contrast image of ovaroids after 2 culture days of ovarian cell suspension, consistent with one or more exemplary embodiments of the present disclosure. Referring to FIG. 2B, after 2 culture days of the ovarian cell suspension, round or oval aggregates as ovaroids 202, with a diameter between about 50 μm and about 100 μm, were formed.

FIG. 2C shows a histological image of an ovaroid by hematoxylin and eosin (H&E) staining, consistent with one or more exemplary embodiments of the present disclosure. FIG. 2D shows a histological image of a magnified portion of the ovaroid by hematoxylin and eosin (H&E) staining, consistent with one or more exemplary embodiments of the present disclosure. Referring to FIGS. 2D and 2C, the histological images of ovaroid 202 indicate the presence of follicular-like structures 206 in section 204 of ovaroid 202. Therefore, ovaroid 202 has a similar structure to a follicular structure of the ovary.

In the next step, after forming the ovaroids, the ovaroids were cultured which allows emersion and radial migration of mOSCs around the ovaroids. In this step, in-vitro derivation and expansion of mOSCs from ovaroids were done. At first, ovaroids were collected using a thin Pasteur pipette and re-plated on a 12-well mouse embryonic fibroblast (MEF)-coated plate with a density of about 5 ovaroids per well. The density of ovaroids in each well may affect the cellular migration and expansion. After optimization, about five ovaroids in one well of a 12-well plate was the optimum condition.

The 12-well plates were pre-coated with mitotically inactivated MEF cells. The MEF cells were treated with mitomycin to obtain mitotically inactivated MEF cells. The medium for culturing the ovaroids was same as the culture medium used for culturing the ovarian cell suspension which was α-MEM supplemented with 10% FBS, 1 mM sodium pyruvate, 1 mM nonessential amino acids, 2 mM 1-glutamine, 1× penicillin-streptomycin, 0.1 mM β-mercaptoethanol, 1× concentrated N2 supplement, 10³ units/ml leukemia inhibitory factor (LIF), 10 ng/ml recombinant human epidermal growth factor (rhEGF), 1 ng/ml basic fibroblast growth factor (bFGF), and 40 ng/ml glial cell-derived neurotropic factor (GDNF).

Three days after plating the ovaroids, round ovaroid-derived cells, with a size between about 5 μm and about 10 μm, migrated and detached from ovaroids and expanded in culture. The ovaroid-derived cells were mOSCs and they were undergoing mitosis which increased the number of cells and formed grape-like clusters. Due to their proliferation, the mOSCs required passaging at confluence about after 3 weeks.

The medium was renewed every 2 or 3 days. The mOSCs were passaged at a split ratio of about 1:2 every 6 or 7 days by enzymatic dissociation using 0.05% trypsin and they were then re-plated on fresh MEF-coated plates. With increased time in culture, almost after 5 passages, the cells proliferated more rapidly and needed to be passaged every 4 or 5 days with a split ratio of 1:3. The proliferation rate of mOSCs was higher than that of hOSCs and the hOSCs were passaged every 30 days with a split ratio of 1:2.

FIG. 2E shows a phase-contrast image of mOSCs derived from re-plated ovaroid after 3 days, consistent with one or more exemplary embodiments of the present disclosure. Referring to FIG. 2E, after 3 days of re-plating and culturing the ovaroids 202, round cells 208 as mOSCs emerged on mitotically inactivated MEF cells 210.

FIG. 2F shows a phase-contrast image of mOSCs derived from re-plated ovaroid after 12 days, consistent with one or more exemplary embodiments of the present disclosure. FIG. 2G shows a phase-contrast image of mOSCs derived from re-plated ovaroid after 21 days, consistent with one or more exemplary embodiments of the present disclosure. Referring to FIG. 2F and 2G, the number of mOSCs 208 were increasing because they were proliferating via mitosis.

FIG. 2H shows a phase-contrast image of a magnified portion of mOSCs derived from re-plated ovaroid after 21 days, consistent with one or more exemplary embodiments of the present disclosure. Referring to FIG. 2H, mOSCs 208 derived from re-plated ovaroid after 21 days were undergoing mitosis.

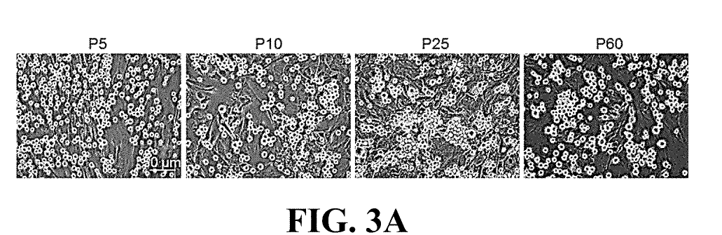

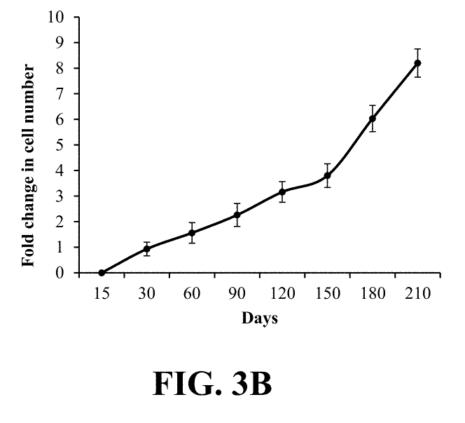

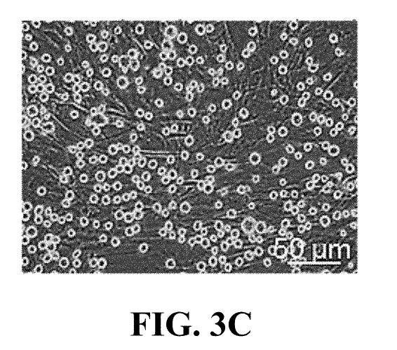

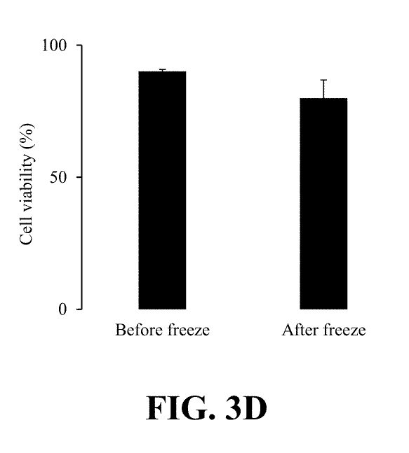

After isolating OSCs in-vitro, they were capable of being expanded in culture for months while being able to sustain their germ cell characteristics. FIG. 3A shows phase-contrast images of expanded mOSCs after 5, 10, 25, and 60 passages on MEF-coated plates, consistent with one or more exemplary embodiments of the present disclosure. Referring to FIG. 3A, the mOSCs may be passaged for 60 passages while being able to maintain their germ cell characteristics.

FIG. 3B shows proliferation pattern of mOSCs during 210 days, consistent with one or more exemplary embodiments of the present disclosure. Referring to FIG. 3B, growth rate of the mOS Cs was low before 15 days or 5 passages, but during further passages, the growth rate of the mOSCs was increasing.

After isolating the mOSCs, the mOSCs were trypsinized, gently mixed with a cryoprotective solution including 90% FBS and 10% dimethyl sulfoxide (DMSO), incubated overnight at a temperature of about −80° C., and then transferred to liquid nitrogen. After cryopreservation, flow cytometry analysis was carried out to assess the effects of cryopreservation on mOSC viability.

FIG. 3C shows a phase-contrast image of mOSCs after thawing, consistent with one or more exemplary embodiments of the present disclosure. The mOSCs were frozen after 12 passages and they propagated 3 passages after thawing. Referring to FIG. 3C, the cryopreservation procedure did not affect cellular morphology and proliferation of the mOSCs. Therefore, it indicates that functional mOSCs may be preserved on a long-term basis.

Cells before and after cryopreservation were incubated with a trypsin-EDTA solution with a concentration of about 0.05% at a temperature of about 37° C. for a time period of about 3 minutes to obtain a single-cell suspension. After that, propidium iodide (PI) was added to the cell suspension just prior to FACS analysis to detect non-viable mOSCs.

FIG. 3D shows FACS results on mOSCs viability before and after cryopreservation after passage 20, consistent with one or more exemplary embodiments of the present disclosure. Referring to FIG. 3D, the viability of the mOSCs was 90.10% before cryopreservation and 79.98% after cryopreservation. Therefore, it indicates that the viability of the mOSCs was not significantly changed after the cryopreservation procedure.

Moreover, immunofluorescence staining and gene expression analysis of mouse ovaroids and mOSCs were done. Gene expression analysis was conducted by isolating and purifying total RNA with a TRIzol reagent, followed by cDNA synthesis. After that, the presence of each indicated mRNA was analyzed by reverse transcription polymerase chain reaction (RT-PCR). Also, immunofluorescence staining was done using a primary antibody, and appropriate secondary antibodies. Also, 4,6-diamidino-2-phenylindole (DAPI) was used for nucleus staining. Immunostaining without primary antibodies was used as a negative control for the cells.

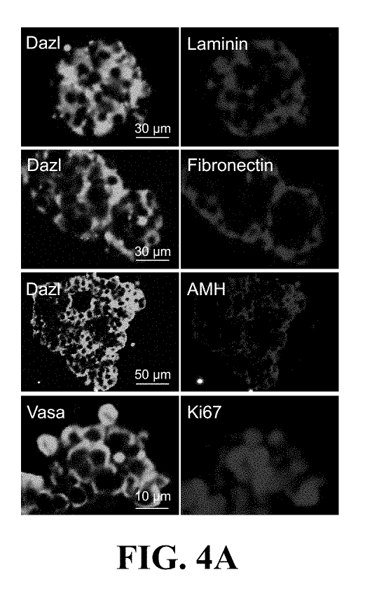

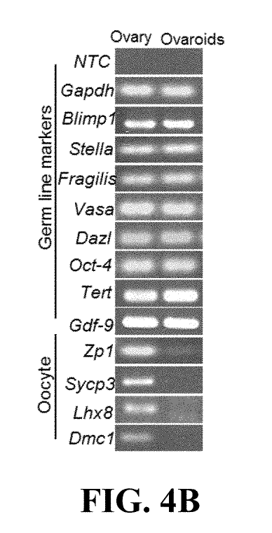

FIG. 4A shows immunofluorescent images of mouse ovaroid sections, consistent with one or more exemplary embodiments of the present disclosure. Immunofluorescence images of the mouse ovaroids determine the localization of the germ and stromal cells in the mouse ovaroid. Referring to FIG. 4A, the cells organized inside the mouse ovaroid, and the ovaroid exhibited a typical structure of an ovary with an outer rim of cells that expressed stromal markers and an internal layer of cells that expressed the germ cell-specific markers of Dazl and Vasa, and a few intermediate cells expressed both germline and stromal markers. The stromal markers are laminin, fibronectin, and anti-Müllerian hormone (AMH). Moreover, the presence of Vasa/Ki67 double-positive cells in the ovaroids confirms the presence of germ cells with a proliferating activity.

FIG. 4B shows results of RT-PCR analysis of ovary versus ovaroids on day 2 of culture, consistent with one or more exemplary embodiments of the present disclosure. RNA sample from the ovary of an adult mouse was used as a positive control. Also, GAPDH mRNA was used as an internal control, and NTC may refer to mock reverse-transcribed RNA samples. Referring to FIG. 4B, results of RT-PCR analysis of the mouse ovaroids confirm that the mouse ovaroids express germ cell-specific markers such as Gapdh, Blimp1, Stella, Fragilis, Vasa, Dazl, Oct-4, Gdf-9, and Tert. Therefore, an organoid culture approach composed of ovarian cells (without oocytes) that mimics the ovarian microenvironment in-vitro would provide a suitable environment for the proliferation and emergence of mitotically active functional germ cells.

Referring again to FIG. 4B, the results of RT-PCR analysis of the mouse ovaroids indicate that the mouse ovaroids do not express oocyte-specific markers such as Zp1, Sycp3, Lhx8, and Dmc1 because the mature oocytes were removed from the ovarian cell suspension used for formation of the ovaroids. Therefore, the mouse ovaroids lack mature oocytes and oocyte-like cells.

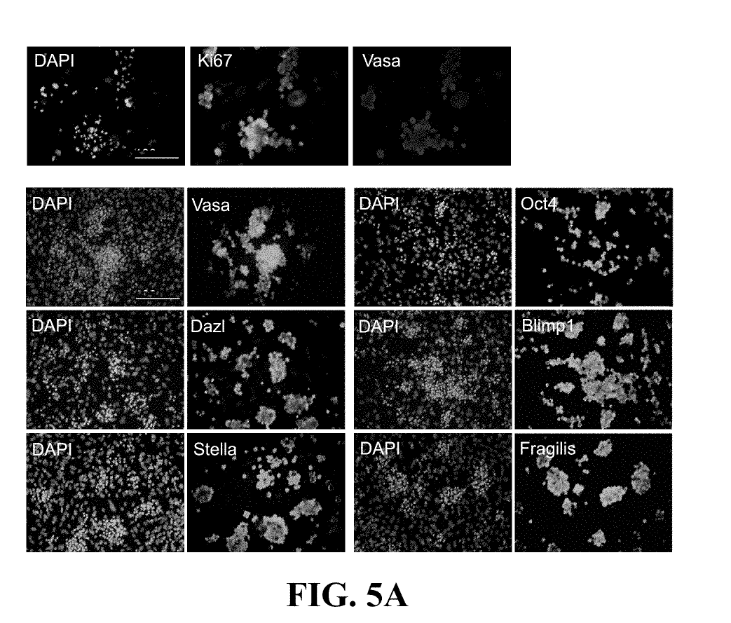

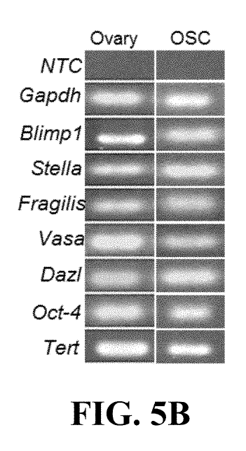

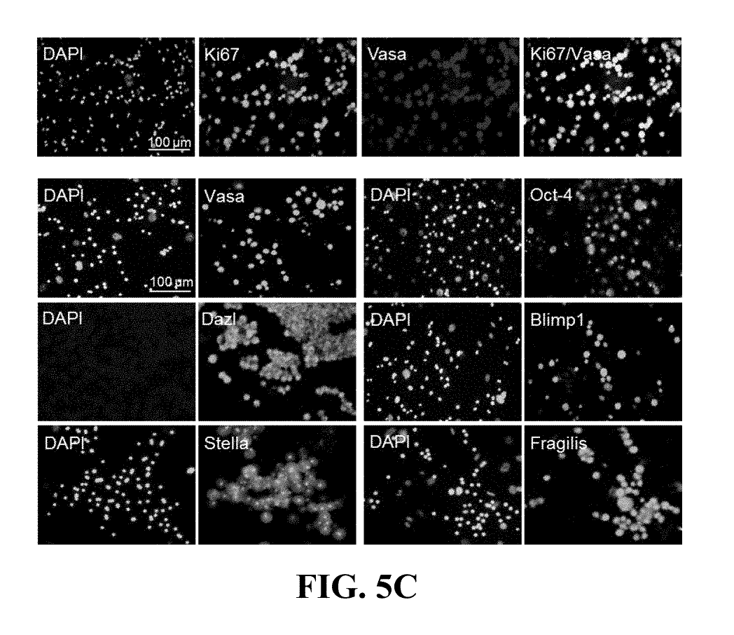

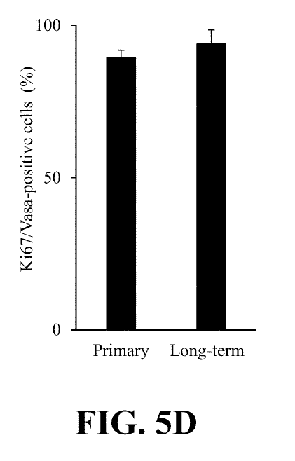

FIG. 5A shows immunofluorescent images of the mOSCs, consistent with one or more exemplary embodiments of the present disclosure. Referring to FIG. 5A, immunofluorescent images of the mOSCs indicates that the mouse germ cells with proliferating activity (mOSCs) express Ki67 (proliferating marker) and Vasa (germ cell-specific marker).

FIG. 5B shows results of RT-PCR analysis of mOSCs for detection of germline stem cell-specific markers after 2 weeks of the ovaroid culture, consistent with one or more exemplary embodiments of the present disclosure. RNA sample from the ovary of an adult mouse was used as a positive control. Also, GAPDH mRNA was used as an internal control, and NTC refers to mock reverse-transcribed RNA samples. Referring to FIG. 5B, results of RT-PCR analysis of mOSCs indicate that the mOSCs express germline stem cell-specific markers such as Blimp1, Stella, Fragilis, Vasa, Dazl, Oct-4, Gdf-9, and Tert. Therefore, the presence of ovaroid-derived mOSCs is confirmed.

FIG. 5C shows immunofluorescent images of the mOSCs for detection of germ cell-specific markers and stem cell-specific marker after 12 passages as a long-term culture, consistent with one or more exemplary embodiments of the present disclosure. Referring to FIG. 5C, long-term culture did not significantly change the expression of the germ cell-specific markers and stern cell-specific markers in mOSCs, and characteristics of the mOSCs remained stable over long-term culture. Also, expressions of germ cell-specific markers and stem cell-specific markers such as such as Ki67, Blimp1, Stella, Fragilis, Vasa, Dazl, and Oct-4 are detected in the mOSCs after long-term culture (passage 12). Moreover, there are Vasa-positive and Ki67-positive mOSCs after long-term culture.

FIG. 5D shows the percentage of Vasa-positive and Ki67-positive mOSCs on day 21 of culture before any passage and after 12 passages as a long-term culture, consistent with one or more exemplary embodiments of the present disclosure. Vasa marker is used as a genii cell-specific marker and Ki67 was used as a marker for actively proliferating cells. Referring to FIG. 5D, the percentage of Vasa-positive and Ki67-positive mOSCs was 89.53% before any passage and 94.11% after 12 passages. Therefore, it may be concluded that characteristics of the mOSCs remained stable over long-term culture as determined by quantification of the relative number of Vasa-positive and Ki67-positive mOSCs. Moreover, the comparison between the percentages of the Vasa-positive and Ki67-positive mOSCs indicates that the number of the mOSCs is increasing in the culture medium during the time.

### Example 2: Isolation Of Human Oogonial Stem Cells

In this example, hOSCs were isolated through the steps of forming an ovarian cell suspension using an adult human ovary, forming ovaroids by culturing the ovarian cell suspension in a three-dimensional culture, and migration of hOSCs around the ovaroids by culturing the ovaroids on a mouse embryonic fibroblast (MEF)-coated plate.

Human ovarian tissue samples were obtained after written informed consent from 8 women in reproductive age, between about 35 and about 45 years old. These women were a candidate for myomectomy, hysterectomy, or tubectomy due to different reasons including uterine fibroids that cause pain, bleeding, or blocked fallopian tubes. All of them signed informed consents, and recorded sex, age, medical history, and relatedness were obtained through a questionnaire at enrollment.

At first, the ovarian cell suspension was formed using the adult human ovary. Biopsies of human ovarian tissues with the size of about 8 cm³ were transferred to αMEM media containing penicillin with a concentration of about 100 U/ml and streptomycin with a concentration of about 100 μg/ml on ice. Biopsies were gently washed several times with HBSS containing antibiotics. Then, the ovarian tissues were dissected and minced by sterile surgical instrument, and rinsed with HBSS. After that, the dissected ovarian tissues underwent enzymatic digestion as described in EXAMPLE 1 for enzymatic digestion of mouse ovarian tissues.

Moreover, forming ovaroids by culturing the ovarian cell suspension and migration of hOSCs around the ovaroids by culturing the ovaroids on a MEF-coated platewere done with the same procedure as described in EXAMPLE 1. Finally, collected supernatants from all enzymatic digestions were filtered through a 40-μm nylon mesh to remove mature oocytes and centrifuged at speed of about 300 g for a time period of about 5 minutes. Then, the supernatant was removed carefully and the ovarian cell suspension was formed by re-suspending clumps of cells in a culture medium. While mature oocytes have inhibitory effects in the ovary, this inhibition was eliminated by removing the mature oocytes from the ovarian cells. Therefore, a permissive and suitable niche for the survival and proliferation of OSCs was provided.

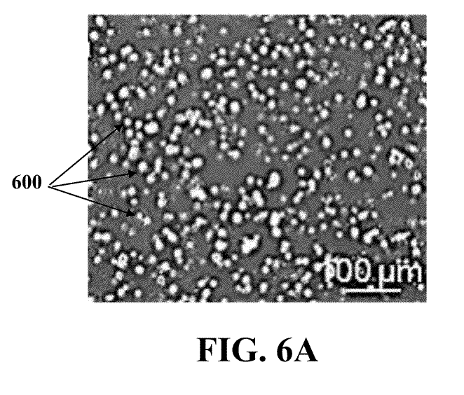

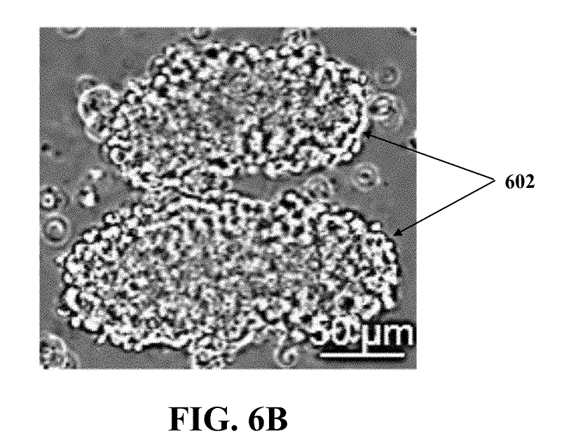

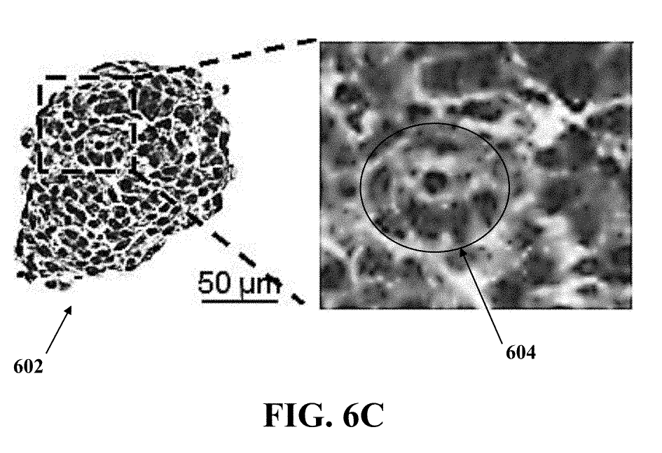

FIG. 6A shows a phase-contrast image of a human ovarian cell suspension, consistent with one or more exemplary embodiments of the present disclosure. Referring to FIG. 6A, the human ovarian cell suspension include homogeneous suspension of human ovarian cells 600 without any aggregations.

In the next step, ovaroids were formed by culturing the ovarian cell suspension. The ovarian cell suspension was cultured under floating conditions on agarose-coated plates as a three-dimensional (3D) culture. Cells of the ovarian cell suspension were seeded on plates pre-coated with about 1% agarose and incubated at a temperature of about 37° C. and at 5% CO₂ atmosphere in a culture medium. After 2 or 3 days of cell culture, round or oval aggregates as ovaroids with a diameter between about 50 μm and about 100 μm were formed.

The culture medium was α-MEM supplemented with 10% FBS, 1 mM sodium pyruvate, 1 mM nonessential amino acids, 2 mM 1-glutamine, 1× penicillin-streptomycin, 0.1 mM β-mercaptoethanol, 1× concentrated N2 supplement, 10³ units/ml leukemia inhibitory factor (LIF), 10 ng/ml recombinant human epidermal growth factor (rhEGF), 1 ng/ml basic fibroblast growth factor (bFGF), and 40 ng/ml glial cell-derived neurotrophic factor (GDNF).

Cell seeding was optimized using different initial cell density on agarose-coated plates between about 1×10⁵ cells per 3 cm² and about 5×10⁵ cells per 3 cm². After optimization, it was found that the density of about 3×10⁵ cells per 3 cm² plate is suitable for aggregate/ovaroid formation at culture time period of about 2 or 3 days. Seeding cells less than about 3×10⁵ per 3 cm² decreased both size and number of aggregates which underwent gradual degeneration. Furthermore, aggregate formation with higher cellular density joined the aggregates together in 24 hours which led to the formation of dense structures.

The ovaroids included both germ cells and somatic cells, and the communication between germ cells and somatic cells are critical for stem cell maintenance and germ cell proliferation. Therefore, the ovaroids served as a well-qualified niche for OSCs because ovaroids as a 3D culture mimic physiological conditions in-vitro by supporting intensive communication between cells and by providing biomechanical and biochemical cues that mediate intercellular functions.

FIG. 6B shows a phase-contrast image of human ovaroids after 2 culture days of human ovarian cell suspension, consistent with one or more exemplary embodiments of the present disclosure. Referring to FIG. 6B, after 2 culture days of the human ovarian cell suspension, round or oval aggregates as human ovaroids, with a diameter between about 50 μm and about 100 μm, were formed.

FIG. 6C shows a histological image of the ovaroid by hematoxylin and eosin (H&E) staining, consistent with one or more exemplary embodiments of the present disclosure. Referring to FIG. 6C, the histological images indicate the presence of follicular-like structures 604 in ovaroid 602. Therefore, ovaroid 602 has a similar structure to the follicular structure of human ovary.

After forming the ovaroids, the ovaroids were cultured which allows emersion and radially migration of hOSCs around the ovaroids. In this step, in-vitro derivation and expansion of hOSCs from ovaroids were done. At first, ovaroids were collected using a thin Pasteur pipette and re-plated on a 12-well mouse embryonic fibroblast (MEF)-coated plate with a density of about 5 ovaroids per well. The density of ovaroids in each well may affect the cellular migration and expansion. After optimization, about five ovaroids in one well of a 12-well plate was the optimum condition.

The 12-well plates were pre-coated with mitotically inactivated MEF cells. The MEF cells were treated with mitomycin to obtain mitotically inactivated MEF cells. The medium for culturing the ovaroids was same as the culture medium used for culturing the ovarian cell suspension which was α-MEM supplemented with 10% FBS, 1 mM sodium pyruvate, 1 mM nonessential amino acids, 2 mM 1-glutamine, 1× penicillin-streptomycin, 0.1 mM β-mercaptoethanol, 1× concentrated N2 supplement, 10³ units/ml leukemia inhibitory factor (LIF), 10 ng/ml recombinant human epidermal growth factor (rhEGF), 1 ng/ml basic fibroblast growth factor (bFGF), and 40 ng/ml glial cell-derived neurotropic factor (GDNF).

Three days after plating the ovaroids, round ovaroid-derived cells with a size between about 5 μm and about 10 μm migrated and detached from ovaroids and expanded in culture. The ovaroid-derived cells were hOSCs, and they were undergoing mitosis which increased the number of cells and formed grape-like clusters. Due to their proliferation, the hOSCs required passaging at confluence about after 3 weeks. The medium was renewed every 2 or 3 days. The proliferation rate of mOSCs was higher than that of hOSCs, and the hOSCs were passaged every 30 days with a split ratio of 1:2.

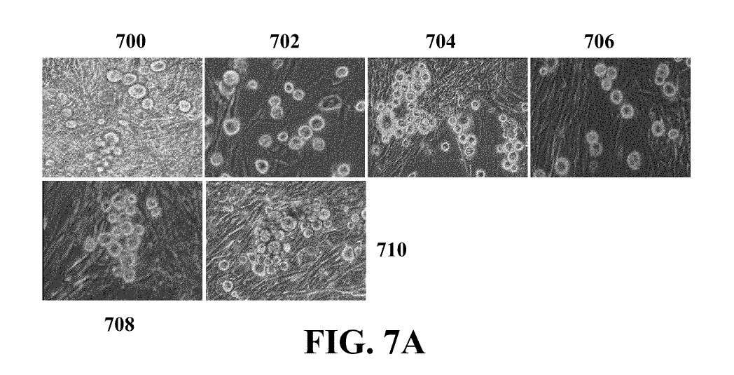

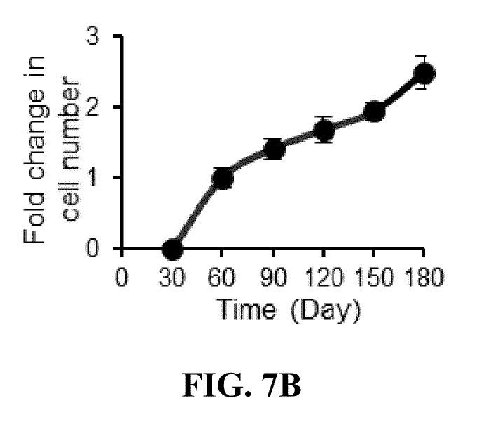

FIG. 7A shows phase-contrast images of expanded hOSCs after 30 days culture 700, 1 passage 702, 3 passages 704, 5 passages 706, 6 passages 708, and 7 passages 710 on MEF-coated plates, consistent with one or more exemplary embodiments of the present disclosure. Referring to FIG. 7A, hOSCs were capable of being expanded in culture for months while being able to sustain their germ cell characteristics.

FIG. 7B shows growth curve of hOSCs, consistent with one or more exemplary embodiments of the present disclosure. Referring to FIGS. 3B and 7B, growth rate of the hOSCs is significantly lower than the growth rate of mOSCs. The fold change in the number of mOSCs was about 6 after 180 days of culture while the fold change in the number of hOSCs was about 3 after 180 days of culture

After isolating the hOSCs, the hOSCs were trypsinized, gently mixed with a cryoprotective solution including 90% FBS and 10% dimethyl sulfoxide (DMSO), incubated overnight at a temperature of about −80° C., and then transferred to liquid nitrogen. After cryopreservation, flow cytometry analysis was carried out to assess the effects of cryopreservation on hOSC viability.

Cells before and after cryopreservation were incubated with a trypsin-EDTA solution with a concentration of about 0.05% at a temperature of about 37° C. for a time period of about 3 minutes to obtain a single-cell suspension. After that, propidium iodide (PI) was added to the cell suspension just prior to FACS analysis to detect non-viable hOSCs.

Moreover, immunofluorescence staining and gene expression analysis of human ovaroids and hOSCs were done. Gene expression analysis was conducted by isolating and purifying total RNA with a TRIzol reagent, followed by cDNA synthesis. After that, the presence of each indicated mRNA was analyzed by conventional RT-PCR. Also, immunofluorescence staining was done using a primary antibody and appropriate secondary antibodies. Also, 4,6-diamidino-2-phenylindole (DAPI) was used for nucleus staining. Immunostaining without primary antibodies was used as a negative control for the cells.

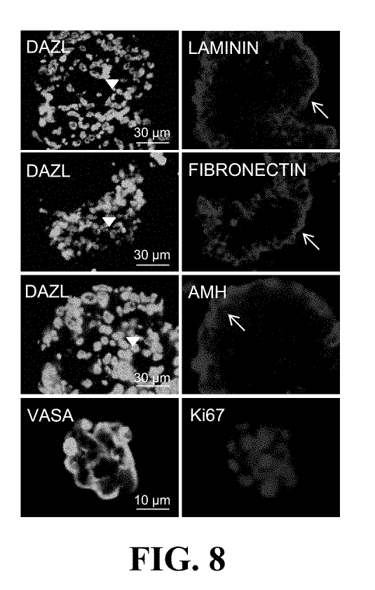

FIG. 8 shows immunofluorescent images of human ovaroid sections for detection of germ cell-specific markers, consistent with one or more exemplary embodiments of the present disclosure. Also, actively proliferating cells were detected using Ki67 marker. Referring to FIG. 8, immunofluorescent images of human ovaroid sections indicate that the cells organized inside the human ovaroid, and the ovaroid exhibited a typical structure of an ovary with an outer rim of cells that expressed stromal markers and an internal layer of cells that expressed the germ cell-specific markers of Dazl and Vasa, and a few intermediate cells expressed both germline and stromal markers. The stromal markers are laminin, fibronectin, and anti-Müllerian hormone (AMH). Moreover, the presence of Vasa/Ki67 double-positive cells in the human ovaroids confirms the presence of germ cells with a proliferating activity.

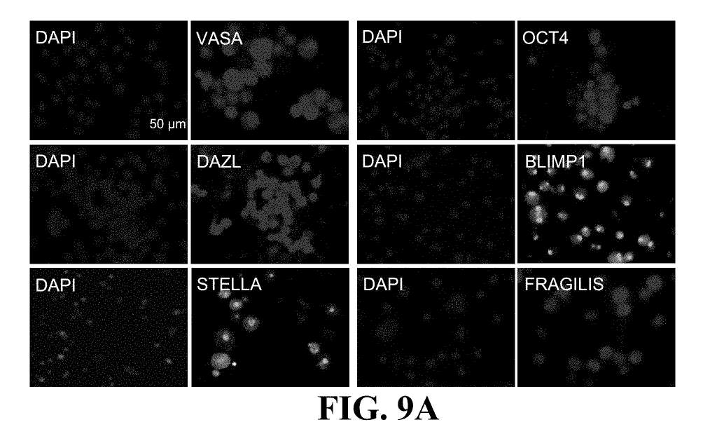

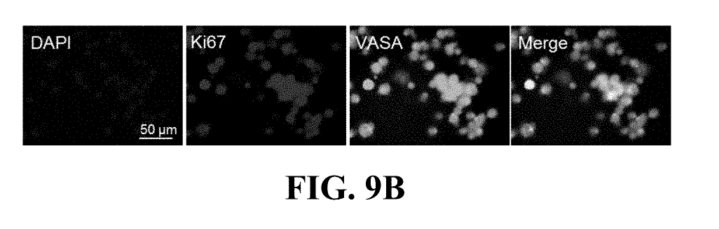

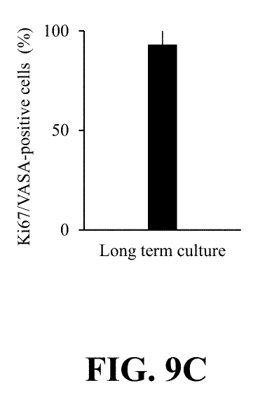

FIG. 9A shows immunofluorescent images of hOSCs for detection of germ cell-specific markers, consistent with one or more exemplary embodiments of the present disclosure. Also, cells were counterstained with 4,6-diamidino-2-phenylindole (DAPI) with blue color to visualize nucleus of the cells. Referring to FIG. 9A, immunofluorescent images of hOSCs indicates that the hOSCs express germ cell-specific markers such as Vasa, Dazl, Blimp1, Oct4, Stella, and Fragilis.

FIG. 9B shows immunofluorescent images of hOSCs for Vasa and Ki67 markers, consistent with one or more exemplary embodiments of the present disclosure. Referring to FIG. 9B, immunofluorescent images of hOSCs after 6 months in culture indicate that the expanded hOSCs concurrently expressed Ki67 and VASA which provided evidence of their proliferative capacity.

FIG. 9C shows percentage of Vasa-positive and Ki67-positive hOSCs after 6 months as a long-term culture, consistent with one or more exemplary embodiments of the present disclosure. Vasa marker is used as a germ cell-specific marker and Ki67 was used as a marker for actively proliferating cells. Referring to FIG. 9C, 93.16% of the isolated hOSCs are actively proliferating cells after 6 months in culture.

### Example 3: In-Vitro Differentiation of OSCs into Oocyte-Like Cells (OLCS)

In this example, mouse and human OSCs isolated in EXAMPLE 1 and EXAMPLE 2 were differentiated into oocyte-like cells (OLCs) in-vitro. During in-vitro expansion of OSCs, spherical OLCs formed in the culture through spontaneous differentiation. However, in order to conduct directed oocyte differentiation, the OSCs were cultured in a differentiation medium. Also, every 2 or 3 days, half of the medium was replaced with a fresh medium.

The differentiation medium contained α-MEM supplemented with 10% FBS, 1 mM sodium pyruvate, 1 mM nonessential amino acids, 2 mM L-glutamine, 1× concentrated penicillin-streptomycin, 0.1 mM β-mercaptoethanol, 1× concentrated N2 supplement, 10 ng/ml rhEGF, a solution of insulin, transferrin, and selenium (ITS) with a concentration of about 5 μl/ml, 0.05 IU follicle-stimulating hormone (FSH), and 0.03 IU luteinizing hormone (LH).

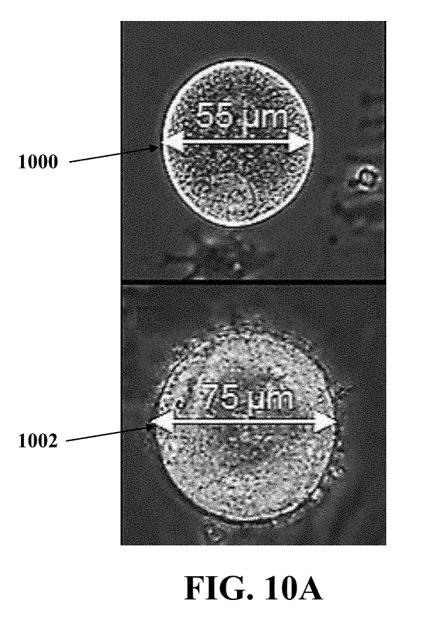

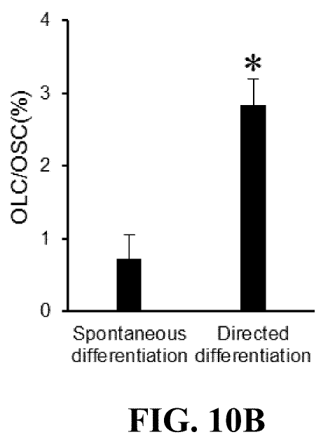

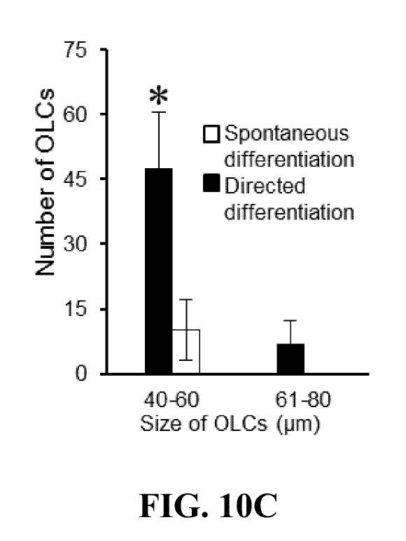

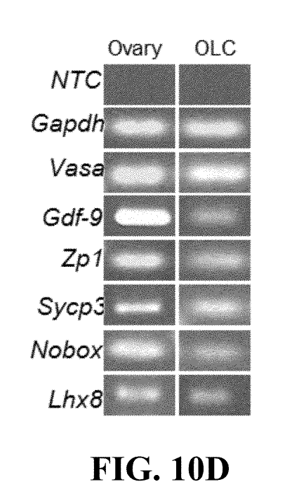

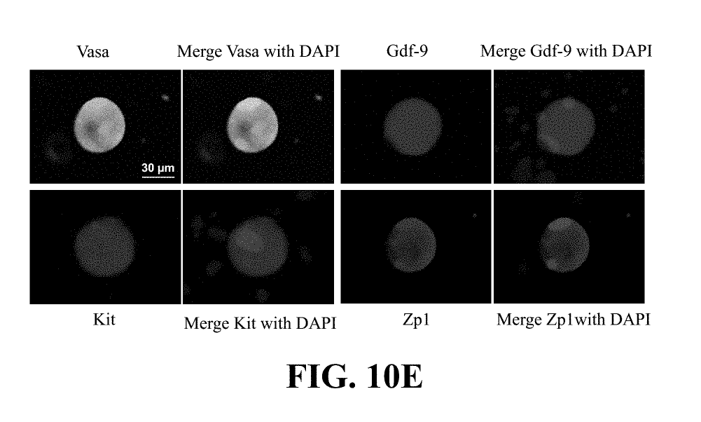

Characteristics of OLCs generated from mOSCs were determined. FIG. 10A shows phase-contrast images of mouse oocyte-like cells (mOLCs), consistent with one or more exemplary embodiments of the present disclosure. Referring to FIG. 10A, mOLC 1000 with a diameter of about 55 μm is formed via spontaneous differentiation of mOSCs in a culture medium and morphologically resembled oocytes. Also, mOLC 1002 with a diameter of about 75 μm is formed via directed differentiation. Referring again to FIG. 10A, number of OLCs formed via directed differentiation is more than the OLCs formed via spontaneous differentiation. Moreover, directed differentiation formed larger OLCs than spontaneous differentiation.

FIG. 10B shows ratio of oocyte-like cells and oogonial stem cells (OLCs/OSCs) in spontaneous differentiation and directed differentiation, consistent with one or more exemplary embodiments of the present disclosure. Referring to FIG. 10B, the ratio of OLCs/OSCs for directed differentiation is about three times more than the ratio of OLCs/OSCs for spontaneous differentiation. Therefore, the differentiation medium in directed differentiation provided a more suitable environment for differentiation of OSCs into OLCs.

FIG. 10C shows size distribution and number of formed OLCs in spontaneous differentiation and directed differentiation, consistent with one or more exemplary embodiments of the present disclosure. Referring to FIG. 10C, the OLCs formed via directed differentiation are more than the OLCs formed via spontaneous differentiation. The OLCs formed via directed differentiation have a diameter with a range from 40 μm to 80 μm, while the OLCs formed via spontaneous differentiation have a diameter with a range from 40 μm to 60 μm. Therefore, directed differentiation of OSCs to OLCs led to significantly larger and more numerous OLCs in culture compared with spontaneously formed OLCs.

FIG. 10D shows mRNA expression levels of oocyte-specific markers in spontaneously differentiated oocyte-like cells (OLCs), consistent with one or more exemplary embodiments of the present disclosure. Referring to FIG. 10D, the spontaneously differentiated OLC expressed all the oocyte-specific markers such as Vasa, Gdf-9, Zp1, Sycp3, Nobox, and Lhx8. Therefore, it indicated that the OSCs had been differentiated into OLCs properly.

FIG. 10E shows immunofluorescence images of spontaneously differentiated oocyte-like cells (OLCs) for detection of oocyte-specific markers, consistent with one or more exemplary embodiments of the present disclosure. Also, DAPI was used for nucleus staining Referring to FIG. 10E, the spontaneously differentiated OLC expressed all the oocyte-specific markers such as Vasa, Gdf-9, Zp1, and Kit. Therefore, it indicated that the OSCs had been differentiated into OLCs properly and expressed oocyte-specific markers in protein level.

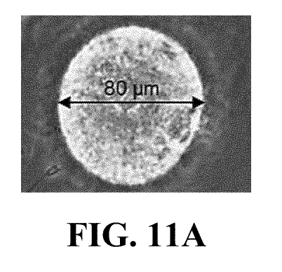

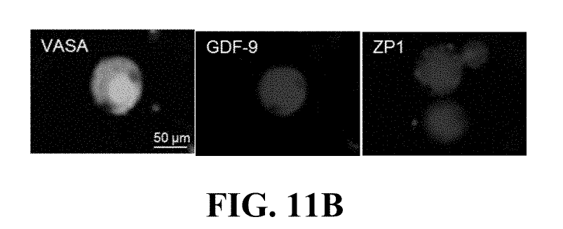

Also, characteristics of OLCs generated from spontaneous differentiation of hOSCs were determined. FIG. 11A shows a phase-contrast image of a human oocyte-like cell (hOLC), consistent with one or more exemplary embodiments of the present disclosure. Referring to FIG. 11A, the hOLC was formed by spontaneously differentiation of hOS Cs and has a diameter of about 80 μm.

FIG. 11B shows immunofluorescence images of spontaneously differentiated oocyte-like cells (OLCs) for detection of oocyte-specific markers, consistent with one or more exemplary embodiments of the present disclosure. Referring to FIG. 11B, the spontaneously differentiated human oocyte-like cells (hOLCs) express oocyte-specific markers such as Vasa, Gdf9, and Zp1. Therefore, it indicated that the hOSCs had been differentiated into hOLCs properly.

### Example 4: Intraovarian Transplantation of OSCs

In this example, intraovarian transplantation and xenotransplantation of OSCs were studied using green fluorescent protein (GFP)-positive mOSCs and labeled hOSCs. The GFP-positive OSCs were isolated using ovarian tissues obtained from transgenic mice that expressed GFP under chicken beta-actin promoter and cytomegalovirus enhancer (pCAG).

At first, intraovarian transplantation of GFP-positive mOSCs was done as follows. Cultured mOSCs were trypsinized and resuspended in a culture medium. The recipient mice were anesthetized with intraperitoneal injections of ketamine with a concentration of about 50 mg/kg and xylazine with a concentration of about 20 mg/kg. Then, about 10⁴ mOSCs were injected into each ovary of the recipients. Microinjection was done using a glass pipette with a tip diameter of about 40 μm.

In order to investigate the functionality of the mOSCs, some mice were sterilized by chemotherapy prior to mOSCs transplantation. Sterilization of mice was done by injecting busulfan with a concentration of about 30 mg/kg body weight and cyclophosphamide with a concentration of about 120 mg/kg body weight in 2-month-old female mice. The transplantation of ovarian cells was performed 2 weeks after chemotherapy treatment.

After between 30 and 60 days of transplantation, the mice were allowed to mate with wild-type C57BL/6 male mice. At the same time, some of the ovaries were harvested, fixed, and processed for immunohistochemical detection of GFP. Ovaries from non-transplanted wild-type female mice served as negative controls for GFP detection.

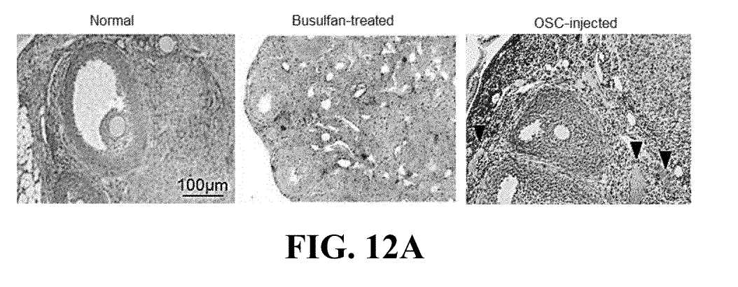

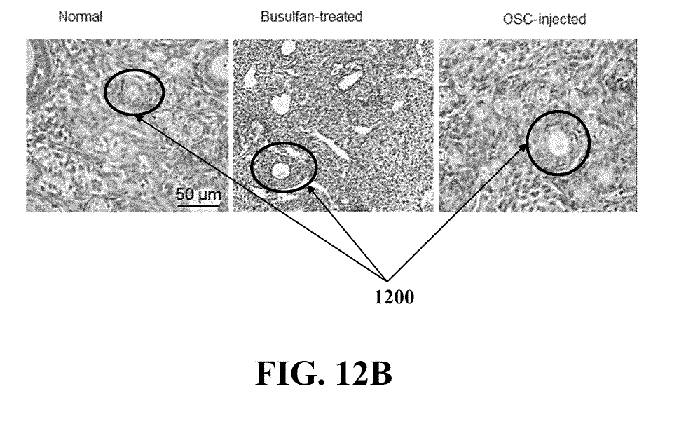

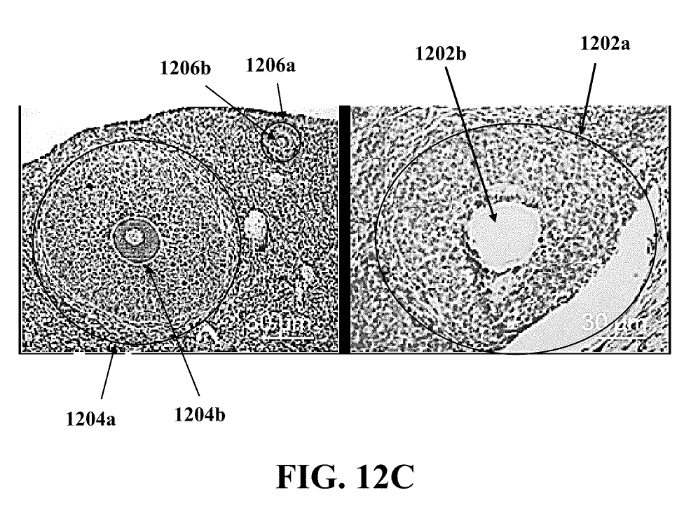

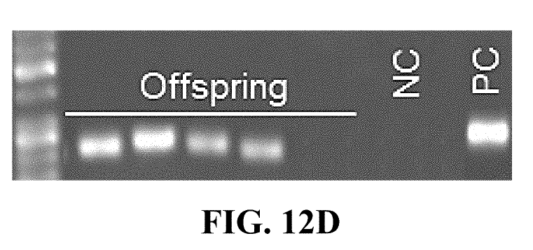

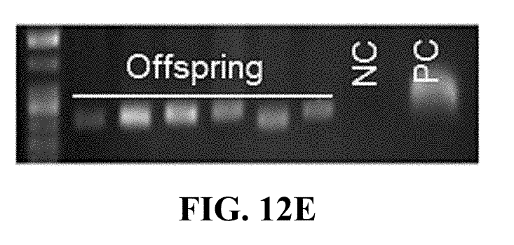

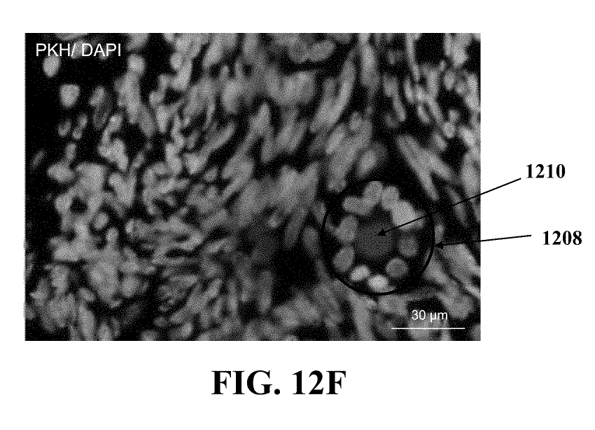

FIG. 12A shows histological images of a normal ovary, busulfan treated ovary, and an ovary with transplanted mOSCs by hematoxylin and eosin (H&E) staining, consistent with one or more exemplary embodiments of the present disclosure. FIG. 12B shows histological images of magnified portions of different ovaries after different treatments by hematoxylin and eosin (H&E) staining, consistent with one or more exemplary embodiments of the present disclosure. Referring to FIGS. 12A and 12B, the comparison between the normal ovary and busulfan-treated ovary show that chemotherapy treatment using busulfan destroys the follicular structure of the ovary. While the ovary with transplanted mOSCs has follicular structures 1200 like a normal ovary, it may be understood that transplanting mOSCs into the ovary provided a suitable environment for regenerating the follicular structure of the ovary after chemotherapy treatment.

FIG. 12C shows histological images of follicles of the mOSCs transplanted ovary at various stages of maturation after immunohistochemistry (IHC) staining, consistent with one or more exemplary embodiments of the present disclosure. The transplanted mOSCs were GFP-positive. Referring to FIG. 12C, follicle 1202a is a secondary follicle with GFP-negative oocyte 1202b, follicle 1204a is a secondary follicle with GFP-positive oocyte 1204b, and follicle 1206a is a primary follicle with GFP-positive oocyte 1206b. While the transplanted mOSCs were GFP-positive, they generated follicular structures 1204a and 1206a with GFP-positive oocytes 1204b and 1206b in the ovary.

Upon transplantation into recipient mice, mOSCs may contribute to the development of new offspring. FIG. 12D shows gel electrophoresis profile for PCR products of offspring derived from transplanted GFP-positive mOSCs following natural mating with wild-type C57BL/6 male mice, consistent with one or more exemplary embodiments of the present disclosure. After mating, 83% (5/6) of the OSC-injected mice produce viable offspring between 4 weeks and 8 weeks after transplantation. Referring to FIG. 12D, results of PCR confirmed the presence of GFP in the genome of the offspring and 27% (5/18) of the F1 progeny were positive for GFP.

FIG. 12E shows gel electrophoresis profile for PCR products of F2 progeny derived from transplanted GFP-positive mOSCs following natural mating with wild-type C57BL/6 male mice, consistent with one or more exemplary embodiments of the present disclosure. Referring to FIG. 12E, results of PCR indicates that all of the second generation (F2 progeny) derived from transplanted GFP-positive mOSCs following natural mating with wild-type C57BL/6 male mice maintained GFP gene in their genome and had genomic stability.

Moreover, the functionality of hOSCs for generating oocyte in-vivo was determined by intraovarian injection of hOSCs and xenotransplantation. Labeling hOSCs was done using PKH-26 GL dye for tracking purposes in-vivo. Intraovarian injection of hOSCs was done as follows. Approximately 10³ labeled hOSCs were resuspended in DMEM solution with 5% FBS and injected into six human ovarian cortical tissue pieces with a size of about 20 mm³.

After that, xenotansplantation of the human ovarian tissue pieces with injected hOSCs was done using nude female mice. Recipient nude female mice were anesthetized and a small incision with a size of about 3 mm was made in the skin on the dorsal midline of the recipient animal, and the human ovarian tissues were inserted to the edge of the incision with 4 grafts per mouse. After two and four weeks of transplantation, xenografts were removed, fixed in 4% paraformaldehyde (PFA), paraffin-embedded, serially sectioned with a thickness of about 6 pm, and mounted onto slides. The slides were de-waxed in xylene and rehydrated through an ethanol gradient. Tissue sections were stained with DAPI for nuclear staining.

FIG. 12F shows immunofluorescence image of human ovarian tissue pieces with injected hOSCs after two weeks of xenotransplantation into nude female mice, consistent with one or more exemplary embodiments of the present. disclosure. Referring to FIG. 12F, the injected PKH-labeled hOSCs involved in the structure of primordial follicle 1208 of the human ovarian tissue pieces and formed PKH-labeled oocyte 1210 through differentiation.

### Example 5: In-Vitro Fertilization of OSCs-Derived Oocytes

In this example, In-vitro fertilization (IVF) of mOSCs-derived oocytes was studied using wild-type adult male NMRI mice between 6 and 8 weeks old for obtaining sperm. At first, between about 30 days and 60 days after mOSCs transplantation, the recipients were treated with 7.5 IU pregnant mare serum gonadotropin (PMSG) and treated with 7.5 IU human chorionic gonadotropin (HCG) after 48 hours of PMSG injection.

Approximately 14 hours after HCG injection, the mice were sacrificed by cervical dislocation. Their oviducts were collected in α-MEM medium supplemented with 10% FBS equilibrated with 5% CO₂ under mineral oil at a temperature of about 37° C. In-vivo matured oocytes inside the ovulated cumulus-oocyte complexes were collected by puncturing the oviducts with a 26G needle and fertilized in-vitro.

IVF was performed in an equilibrated T6 medium that contained 0.4% bovine serum albumin (BSA) using sperm capacitated in the same medium for a time period of about 1 hour. Oocytes were left with sperm in the fertilization medium for a time period of about 4 hours. After that, the fertilized oocytes were washed and subsequently moved to an equilibrated G1 embryo medium, followed by incubation at a temperature of about 37° C. with 5% CO₂ for a time period of about 24 hours.

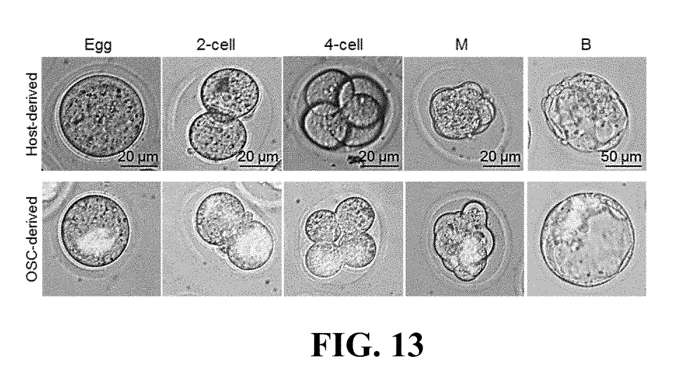

Light and fluorescence microscopic examinations were performed every 24 hours for a total of 144 hours to monitor embryo development. FIG. 13 shows microscopic images of embryo development of a mOS Cs-derived oocyte and a host-derived oocyte, consistent with one or more exemplary embodiments of the present disclosure. The host-derived oocyte was used as a control group. Also, the presence of GFP in the embryo indicated that this embryo was produced from mOSCs which were GFP-positive. Referring to FIG. 13, the mOSC-derived oocyte produce a 2-cell embryo, a 4-cell embryo, a morula, and a blastocyst during its embryo development. This result confirmed that the mOSCs isolated from mouse ovaroids and expanded in-vitro could sustain their ability for integration into their original microenvironment and contribute to neo-oogenesis in-vitro.

### Example 6: Teratoma Formation Assay

In this example, teratoma formation assay of the OSCs was studied by subcutaneous injection of about 10⁵ mOSCs into the neck area of 3 nude female mice. Control groups were age-matched nude female mice injected at the same site with mouse embryonic stem cells (mESCs). Mice were monitored weekly for teratoma formation up to 3 months. After that, histological sections of the mOSCs-injected sites of the neck area were obtained for further analysis.

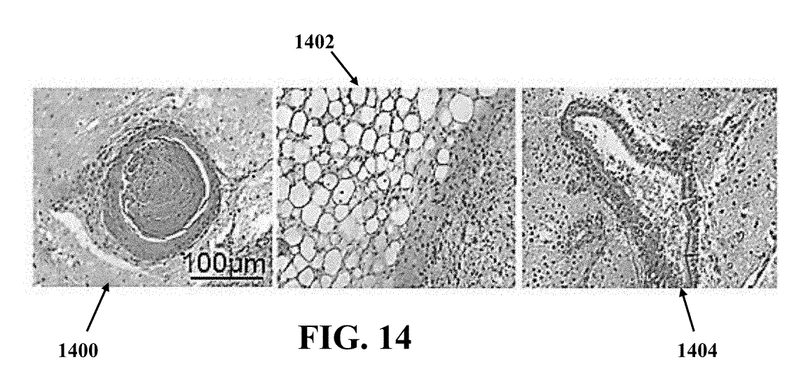

FIG. 14 shows histological images of the mOSCs-injected sites of the neck area of mice stained with hematoxylin and eosin (H&E) staining, consistent with one or more exemplary embodiments of the present disclosure. Referring to FIG. 14, no tumor was observed 3 months after the mOSC transplantation, whereas mESCs formed tumors one month after the transplantation. The derivatives of the three germ layers such as derivation of ectoderm 1400, derivation of mesoderm 1402, and derivation of endoderm 1404 were observed in histological sections of ESC-injected sites.

### Example 7: Evaluation of Endocrine Functions of Ovaroids

One of the common analysis for evaluating the functionality of organoids such as ovaroids is measuring their sensitivity to different hormones. In this example, endocrine functions of the ovaroids were evaluated by measuring levels of secreted 17 β-estradiol and inhibin from ovaroids in the presence of gonadotropin hormones of follicle-stimulating hormone (FSH) and luteinizing hormone (LH).

At first, ovarian cells were cultured in the presence and absence of 0.01 IU FSH and 7.5 IU LH for 2 days under 3D culture conditions in the basic medium including α-MEM medium with LIF, EGF, bFGF, and GDNF. Then, the culture media was collected to assess the secretion of 17 β-estradiol and inhibin hormones according to an ELISA kit.

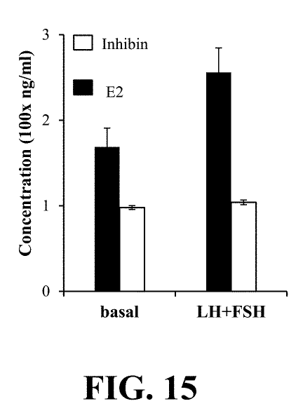

FIG. 15 shows levels of β-estradiol (E2) and inhibin produced by ovaroids in the presence or absence of FSH and LH, consistent with one or more exemplary embodiments of the present disclosure. Referring to FIG. 15, hormone synthesis analysis in the ovaroids showed a significant increase in the secretion of β-estradiol (E2) and inhibin from ovaroids in response to gonadotropins (FSH and LH hormones). Therefore, it may be concluded that the ovaroids had an acceptable endocrine function.

### Example 8: Alkaline Phosphatase Activity of Ovaroid-Derived OSCs

Although OSCs express numerous stem cell and primitive germ cell-specific markers, these cells are distinct from the other types of pluripotent stem cells. While OSCs express low levels of alkaline phosphatase (AP) in comparison with ESCs, the AP activity of cells may be used as an indicator for distinguishing OSCs from ESCs. In this example, an alkaline phosphatase staining kit was used to detect alkaline phosphatase (AP) activity of ovaroid-derived OSCs.

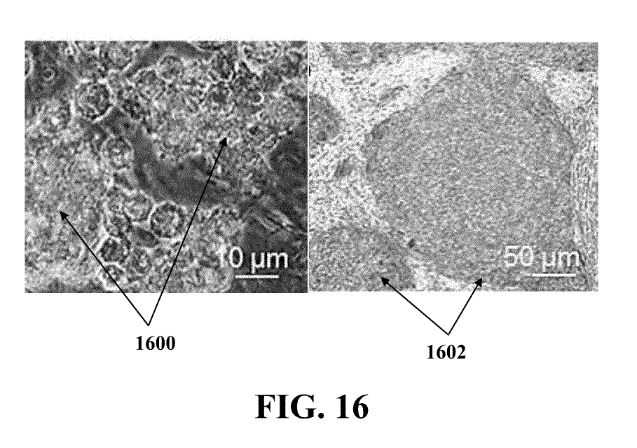

Embryonic stem cells (ESCs) were used as the positive control. FIG. 16 shows alkaline phosphatase staining (AP) of ovaroid-derived OSCs an ESCs, consistent with one or more exemplary embodiments of the present disclosure. Referring to FIG. 16, mOSCs 1600 had very low AP activity because they were stained very weak, whereas ESCs 1602 as a positive control group was highly stained and had a high level of AP activity.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in the light of the attached claims and their equivalents. Also, various modifications and changes may be made within the scope of the attached claims.

What is claimed is:

1. A method for isolating oogonial stem cells (OSCs), comprising:
forming an ovarian cell suspension from an ovary;
forming ovaroids by culturing the ovarian cell suspension in a three-dimensional culture; and
migrating the OSCs around the ovaroids by culturing the ovaroids on a mouse embryonic fibroblast (MEF)-coated plate.

2. The method according to claim 1, wherein culturing the ovarian cell suspension in the three-dimensional culture comprises culturing the ovarian cell suspension using one of an agarose-coated plate, a hanging drop, or a microwell.

3. The method according to claim 1, wherein forming the ovaroids by culturing the ovarian cell suspension comprises culturing the ovarian cell suspension for a period of time between 2 days and 3 days.

4. The method according to claim 1, wherein forming the ovaroids by culturing the ovarian cell suspension comprises culturing the ovarian cell suspension with a density between 2500 cell/cm² and 500000 cell/cm².

5. The method according to claim 1, wherein forming the ovaroids by culturing the ovarian cell suspension comprises culturing the ovarian cell suspension in a medium comprising a growth factor.

6. The method according to claim 5, wherein the growth factor comprises one of epidermal growth factor (EGF), basic fibroblast growth factor (bFGF), glial cell line-derived neurotrophic factor (GDNF), leukemia inhibitory factor (LIF), or combinations thereof.

7. The method according to claim 1, wherein forming the ovarian cell suspension from the ovary comprises removing mature oocytes from the ovarian cell suspension using a filter with a 40 μm filter mesh.

8. The method according to claim 1, wherein the ovaroids have a diameter between 50 μm and 100 μm.

9. The method according to claim 1, wherein the ovaroids have a round shape or an oval shape.

10. The method according to claim 1, wherein migrating the OSCs around the ovaroids by culturing the ovaroids comprises culturing the ovaroids with a density of 1 ovaroid/cm².

11. The method according to claim 1, wherein migrating the OSCs around the ovaroids by culturing the ovaroids comprises culturing the ovaroids for a time period between one week and three weeks.

12. The method according to claim 1, wherein migrating the OSCs around the ovaroids by culturing the ovaroids comprises culturing the ovaroids in a medium comprising a growth factor.

13. The method according to claim 12, wherein the growth factor comprises one of epidermal growth factor (EGF), basic fibroblast growth factor (bFGF), glial cell line-derived neurotrophic factor (GDNF), leukemia inhibitory factor (LIF), or combinations thereof.

14. The method according to claim 1, wherein the OSCs has a germ cell-specific marker comprising Vaza, Dazl, Oct4, Fragilis, Stella, and Blimp-1.

15. The method according to claim 1, wherein the OSCs comprises one of mouse OSCs (mOSCs) or human OSCs (hOSCs).
